(19) United States
(12) Patent Application Publication
(10) Pub. No.: US 2023/0029269 A1
(43) Pub. Date: Jan. 26, 2023

(54) METHOD, APPARATUS, AND SERVER FOR COLLECTING INFORMATION

(71) Applicant: Shenzhen Fugui Precision Ind. Co., Ltd., Shenzhen (CN)

(72) Inventor: Chien-Chang LIN, New Taipei (TW)

(21) Appl. No.: 17/382,509

(22) Filed: Jul. 22, 2021

(51) Int. Cl. G07C 9/25; G07C 9/27; G06F 16/955; G06F 3/0481

(52) U.S. Cl. CPC G07C 9/257; G07C 9/27; G06F 16/9554; G06F 3/04817

(57) ABSTRACT

An apparatus for collecting information communicating with a server and a collecting device includes scanning a bar code and transmit identification information of the apparatus to the server; the bar code is configured to link to a webpage of the server; acquiring biological characteristic information of a user of the apparatus collected by the collecting device; acquiring the identification information and the biological characteristic information matched by the server; and displaying the identification information and the biological characteristic information synchronized with the server. A method and a server for collecting information are also disclosed.

### FIELD

The subject matter herein generally relates to information processing technology, and particularly to a method, an apparatus, and a server for collecting information.

### BACKGROUND

Nowadays, due to virus pandemic, regional protection and local examination is important for stopping the spreading of the pandemic. A safety performance requirement of locality and the regional protection is increasing, an in-and-out management for the people exiting and entering the region is no longer limited to identity authentication, collecting biological characteristic information (such as body temperature, fingerprints, iris, facial characteristic, and/or body characteristic) of the people is also needed. By collecting biological characteristic information of the people exiting and entering the region, an analysis, a protection alarm, and an notification can be processed rapidly. However, after collecting biological characteristic information of the people exiting and entering the region, a process of matching the collected biological characteristic information and the identity information needs to be operated artificially, to record information for the people in a server. This way of procedure needs much time, often blocking the entrance when occurring at rush hours.

### BRIEF DESCRIPTION OF THE DRAWINGS

Many aspects of the disclosure can be better understood with reference to the following drawings. The components in the drawings are not necessarily drawn to scale, the emphasis instead being placed upon clearly illustrating the principles of the disclosure. Moreover, in the drawings, like reference numerals designate corresponding parts throughout the several views.

FIG. 1 is a schematic view of at least one embodiment of a system for collecting information.

FIG. 2 is a schematic view of at least one embodiment of the system of FIG. 1 showing information interaction.

FIG. 3 illustrates a flowchart of at least one embodiment of a method for collecting information.

FIG. 4 illustrates a flowchart of another embodiment of a method for collecting information.

FIG. 5 is a schematic view of at least one embodiment of the system of FIG. 1 applied in an information-collecting scenario.

FIG. 6 is a schematic view of another embodiment of the system of FIG. 1 applied in an information-collecting scenario.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts have been exaggerated to better illustrate details and features of the present disclosure.

The present disclosure, including the accompanying drawings, is illustrated by way of examples and not by way of limitation. Several definitions that apply throughout this disclosure will now be presented. It should be noted that references to “an” or “one” embodiment in this disclosure are not necessarily to the same embodiment, and such references mean “at least one.”

Furthermore, the term “module”, as used herein, refers to logic embodied in hardware or firmware, or to a collection of software instructions, written in a programming language, such as Java, C, or assembly. One or more software instructions in the modules can be embedded in firmware, such as in an EPROM. The modules described herein can be implemented as either software and/or hardware modules and can be stored in any type of non-transitory computer-readable medium or another storage device. Some non-limiting examples of non-transitory computer-readable media include CDs, DVDs, BLU-RAY, flash memory, and hard disk drives. The term “comprising” means “including, but not necessarily limited to”; it in detail indicates open-ended inclusion or membership in a so-described combination, group, series, and the like.

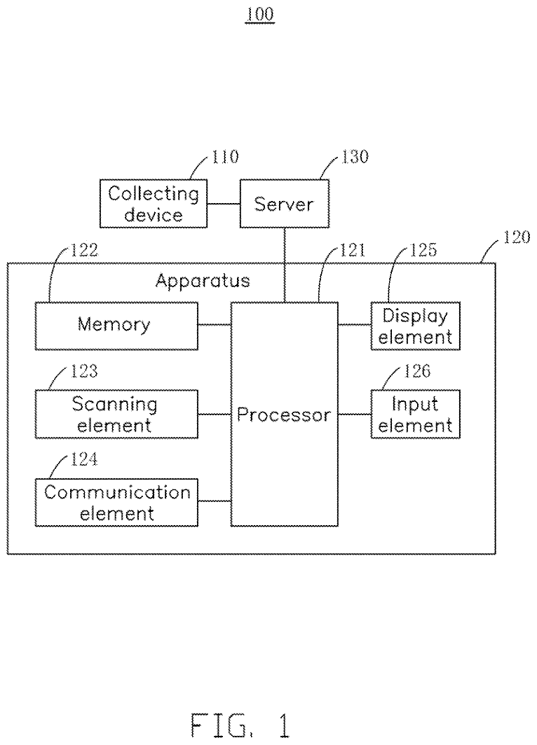

FIG. 1 illustrates at least one embodiment of a system 100 for collecting information.

The system 100 includes a collecting device 110, an apparatus 120, and a server 130. The server 130 is communicating with the collecting device 110 and the apparatus 120.

In at least one embodiment, the communication connection of the server 130, the collecting device 110, and the apparatus 120 can be in cable connection or in wireless connection. The cable connection can be in wired transmission medium (such as optical fiber or copper wire) connection. The wireless connection can be in wireless transmission medium (such as WI-FI, BLUETOOTH, or cellular network like 2G//3G/4G/5G) connection.

In at least one embodiment, the collecting device 110 can be a non-contact collecting device, such as an infrared thermometer, a thermal infrared imager, a laser thermometer, and/or an ultrasonic thermometer, etc. Collecting biological characteristic information by the non-contact collecting device can decrease a risk of contact infection for the virus pandemic.

The apparatus 120 includes a processor 121, a memory 122, a scanning element 123, a communication element 124, a display element 125, and an input element 126. The processor 121, the memory 122, and the elements 123, 124, 125, 126 may be electrically connected to each other through a bus.

In other embodiments, the apparatus 120 can include more elements, modules, components, circuits, chips, functional blocks, and/or arrangements. The elements, devices, apparatus, and systems shown in the figures can be hardware, software, or a combination of hardware and software.

In at least one embodiment, the processor 121 can be a central processing unit (CPU), a microprocessor, a digital signal processor (DSP), an application specific integrated circuit (ASIC), a field-programmable gate array (FPGA), a data processor chip, a programable logic device (PLD), a discrete gate/transistor logic device, or a discrete hardware component.

In at least one embodiment, the memory 122 can include various types of non-transitory computer-readable storage mediums. For example, the memory 122 can be an internal storage system, such as a flash memory, a random access memory (RAM) for the temporary storage of information, and/or a read-only memory (ROM) for permanent storage of information. The memory 122 can also be an external storage system, such as a hard disk, a storage card, or a data storage medium.

The scanning element 123 is configured to scan bar codes to be linked to a webpage of the server 130. The bar code includes one dimensional code, two-dimensional code, and/or quick response code (QR code). The bar code can be generated by the server 130, one bar code is corresponding to one webpage.

In at least one embodiment, the bar code generated by the server 130 can be printed out and shown at the entrance of the protection region for being scanned by the apparatus 120. In another embodiment, a display device can be placed at the entrance of the protection region for showing the bar code obtained from the server 130 for being scanned by the apparatus 120.

The communication element 124 is communicating with the server 130 for exchange of information.

In at least one embodiment, the communication element 124 is configured to transmit identification information of the apparatus 120 to the server 130. The identification information includes an identification code or a serial number of the apparatus 120.

The communication element 124 can provide wireless communication in the apparatus 120. The wireless communication can be but is not limited to wireless local area networks (WLAN) (such as wireless fidelity, WI-FI networks), BLUETOOTH (BT), global navigation satellite system (GNSS), frequency modulation (FM), near field communication (NFC), infrared (IR), etc.

The display element 125 is configured to display information. The display element 125 is further configured to display various interfaces, such as media content interface, call out interface, messenger interface, gaming interface, graph interface, according to user's operations on the apparatus 120.

In at least one embodiment, the display element 125 is configured to display the webpage of the server 130.

The display element 125 can be but is not limited to liquid crystal display (LCD), thin film transistor liquid crystal display (TFT-LCD), light-emitting diode (LED), organic light-emitting diode (OLED), active-matrix organic light-emitting diode (AMOLED), flexible display, curved display, and three-dimensional display.

The input element 126 is configured to receive input instruction from the user to control the apparatus 120. The input element 126 is further configured to receive touch input instruction from the user. The input element 126 may include a touch panel. The touch panel can be at least one category of capacitive, resistive, infrared, and ultrasonic for identifying the touch input instruction. In addition, the touch panel further includes control circuit. When the touch panel is the capacitive type, the touch panel can receive physical touch input or proximity input. The touch panel further includes a touch layer for responding touch reaction to the user. The input element 126 can transmit input signal to the processor 121 according to the received touch input instruction.

The input element 126 may include a digital stylus sensor, keys or an ultrasonic input device. The digital stylus sensor can receive same input instruction from the user, or receive input instruction by an independent identifying board. The keys include physical buttons, optical keys, or keyboards. The ultrasonic input device can be an input device generating ultrasonic signals and wirelessly identifying sonic wave by a microphone (not shown) of the apparatus 120.

In at least one embodiment, when the display element 125 displays the webpage of the server 130, the input element 126, in response to a touch or a click by the user, and transmits a signal to the processor 121. The processor 121 determines whether the identification information displayed in the webpage and the biological characteristic information are normal according to the signal. The identification information and the biological characteristic information are normal includes the identification information and the biological characteristic information are matching, and the biological characteristic information is within a normal range.

For instance, when the identification information is an identification code of the apparatus 120, the biological characteristic information is a body temperature value, and when the body temperature value displayed in the webpage is within a normal body temperature range, which can be but is not limited to 36-37 □, the body temperature value is taken from the user of the apparatus 120, and when the identification code of the apparatus 120 is same as an identification code displayed in the webpage, then the identification information and the biological characteristic information are determined to be normal. When the body temperature value displayed in the webpage is outside the normal body temperature range, or the body temperature value is not from the user of the apparatus 120, or when the identification code of the apparatus 120 is different from the identification code displayed in the webpage, then the identification information and the biological characteristic information are determined to be abnormal.

In at least one embodiment, when the identification information and the biological characteristic information are determined to be normal, the server 130 stores the identification information and the biological characteristic information, and refreshes the webpage, to indicate the user of the apparatus 120 corresponding to the identification information is allowed to enter the protection region. When the identification information and the biological characteristic information are determined to be abnormal, the server 130 generates abnormal alarm to indicate that the user of the apparatus 120 corresponding to the identification information is not allowed to enter the protection region.

The display element 125 and the input element 126 can be integrated. For instance, the apparatus 120 may include a touch screen, which can execute both the input function and the display function. The touch screen can detect touch input on a surface of the touch screen from the user, such as long press/short press touch input, single touch/multi-touch input, touch posture input (such as dragging), etc. When the touch screen detects touch input on the surface of the touch screen from the user, the touch screen further detects a coordinate of the touch and transmits the detected coordinate of the touch to the processor 121.

The touch screen can be configured to convert a pressure on a specific part of the surface of the touch screen or a capacitance change of the specific part of the surface of the touch screen into electrical input signal. The touch screen can be configured to detect touch pressure according to a touch category, a touch position, and a touch area. When the touch screen detects a touch input, a signal corresponding to the touch input can be transmitted to a touch controller (not shown). The touch controller can process one or more signals and transmit corresponding data to the processor 121.

The apparatus 120 can be, but is not limited to a smart phone, a tablet computer, a personal computer (PC), a personal digital assistant (PDA), a camera, and a wearable device.

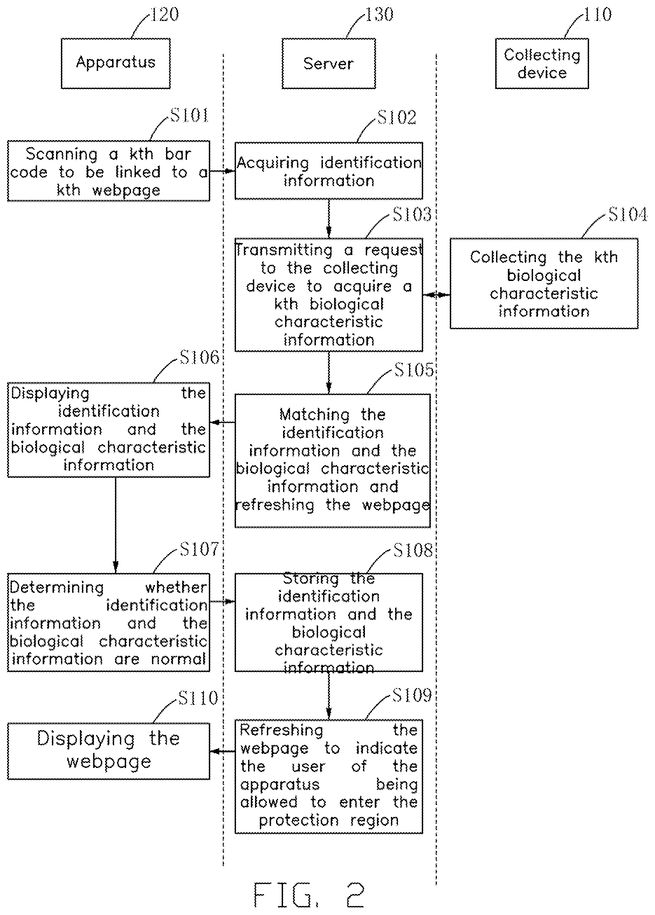

FIG. 2 illustrates a schematic view of at least one embodiment of the system 100 for collecting information showing information interaction.

Referring to FIG. 2, the information interaction process includes:

At block S101, the apparatus 120 scans a kth bar code, to be linked to a kth webpage.

Wherein, k is a positive integer.

At block S102, the server 130 acquires identification information.

In at least one embodiment, the apparatus 120 transmits the identification information to the server 130. The identification information includes an identification code or a serial number of the apparatus 120.

Wherein, the kth webpage displays the identification information.

At block S103, the server 130 transmits a request to the collecting device 110 to acquire kth biological characteristic information.

Wherein, the kth biological characteristic information means biological characteristic information of the user of the apparatus 120. The biological characteristic information includes but is not limited to body temperature, fingerprints, iris, facial characteristic, and/or body characteristic.

At block S104, the collecting device 110 collects the kth biological characteristic information and transmits the biological characteristic information to the server 130.

In at least one embodiment, the collecting device 110 can be a non-contact collecting device that can collect biological characteristic information by at least one way of infrared (IR), radio frequency identification (RFID), or near field communication (NFC).

At block S105, the server 130 matches the identification information and the biological characteristic information, and refreshes the webpage, to di splay the identification information and the biological characteristic information on the webpage.

In at least one embodiment, the matching the identification information and the biological characteristic information includes: establishing a corresponding relationship of the identification information and the user of the apparatus 120; establishing a corresponding relationship of the user of the apparatus 120 and corresponding biological characteristic information; and establishing a corresponding relationship of the identification information and the biological characteristic information.

At block S106, the apparatus 120 displays the identification information and the biological characteristic information.

In at least one embodiment, the apparatus 120 scans the kth bar code to acquire a kth website. The kth website can be linked to the kth webpage of the server 130. The apparatus 120 can be linked to the kth web site by a browser application, and a content of the kth webpage can be displayed on an interface of the browser application. The content of the kth webpage includes the identification information and the biological characteristic information.

At block S107, the apparatus 120, in response to a confirmation operation, determines whether the identification information displayed in the webpage and the biological characteristic information are normal.

In at least one embodiment, the kth webpage displays a confirmation icon. When the confirmation icon detects a confirmation operation from the user, the apparatus 120 generates a determining instruction, to determine whether the identification information displayed in the webpage and the biological characteristic information are normal, and transmits the determining instruction to the server 130. The confirmation operation includes touch or click on the confirmation icon.

At block S108, the server 130 stores the identification information and the biological characteristic information.

In at least one embodiment, the server 130, in response to the determine instruction, stores the identification information and the biological characteristic information in a key-value form. The key (or value) in the key-value form can be the identification information or the biological characteristic information.

At block S109, the server 130 refreshes the webpage to indicate that the user of the apparatus 120 being allowed to enter the protection region.

In at least one embodiment, the kth webpage displays an indication window. The indication window includes indication information indicating that the user of the apparatus 120 being allowed or not allowed to enter the protection region.

At block S110, the apparatus 120 displays the webpage.

In at least one embodiment, the apparatus 120 and the server 130 synchronously refresh the webpage. The webpage displays the identification information, the biological characteristic information, and the indication information.

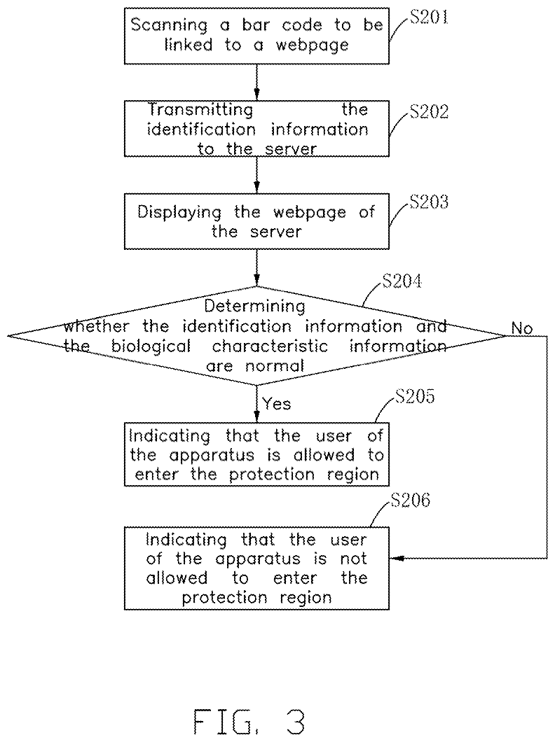

FIG. 3 illustrates a flowchart of at least one embodiment of a method for collecting information. The method is provided by way of example, as there are a variety of ways to carry out the method. Each block shown in FIG. 3 represents one or more processes, methods, or subroutines carried out in the example method. Furthermore, the illustrated order of blocks is by example only and the order of the blocks can be changed. Additional blocks may be added or fewer blocks may be utilized, without departing from this disclosure. The example method can begin at block S201.

At block S201, scanning a bar code to be linked to a webpage.

In at least one embodiment, one bar code is corresponding to one webpage. The apparatus 120 scans a bar code, to be linked to a webpage of the server 130, to display content of the webpage.

At block S202, transmitting the identification information to the server 130.

In at least one embodiment, the identification information includes an identification code or a serial number of the apparatus 120.

At block S203, displaying the webpage of the server 130.

In at least one embodiment, the apparatus 120 displays the webpage of the server 130. The webpage shows the identification information and the biological characteristic information. The biological characteristic information means biological characteristic information of the user of the apparatus 120.

At block S204, determining whether the identification information and the biological characteristic information are normal. If the identification information and the biological characteristic information are determined to be normal, the method goes to block S205. If the identification information and the biological characteristic information are determined to be abnormal, the method goes to block S206.

In at least one embodiment, the server 130 displays a confirmation icon in the webpage. When the confirmation icon detects a confirmation operation from the user, the apparatus 120 determines that the identification information and the biological characteristic information are normal. When the confirmation icon does not detect any confirmation operation from the user, the apparatus 120 determines that the identification information and the biological characteristic information are abnormal. The confirmation operation includes touch or click on the confirmation icon.

In another embodiment, the server 130 displays a confirmation icon and a negative icon in the webpage. When the confirmation icon detects a confirmation operation from the user, the apparatus 120 determines that the identification information and the biological characteristic information are normal. When the negative icon detects a negative operation from the user, the apparatus 120 determines that the identification information and the biological characteristic information are abnormal. The negative operation includes touch or click on the confirmation icon.

At block S205, indicating the user of the apparatus 120 is allowed to enter the protection region.

In at least one embodiment, the server 130 displays first indication information on the webpage. The first indication information indicates that the user of the apparatus 120 is allowed to enter the protection region.

At block S206, indicating the user of the apparatus 120 is not allowed to enter the protection region.

In at least one embodiment, the server 130 displays second indication information on the webpage. The second indication information indicates that the user of the apparatus 120 is not allowed to enter the protection region.

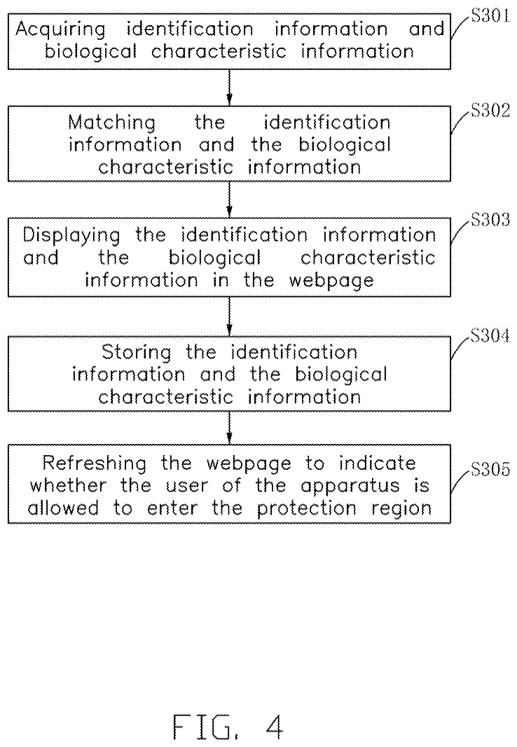

FIG. 4 illustrates a flowchart of another embodiment of a method for collecting information. The method is provided by way of example, as there are a variety of ways to carry out the method. Each block shown in FIG. 4 represents one or more processes, methods, or subroutines carried out in the example method. Furthermore, the illustrated order of blocks is by example only and the order of the blocks can be changed. Additional blocks may be added or fewer blocks may be utilized, without departing from this disclosure. The example method can begin at block S301.

At block S301, acquiring identification information and biological characteristic information.

In at least one embodiment, the server 130 acquires the identification information of the apparatus 120 from the apparatus 120, and acquires the biological characteristic information of the user of the apparatus 120 from a collecting device 110.

At block S302, matching the identification information and the biological characteristic information.

In at least one embodiment, the matching the identification information and the biological characteristic information includes: establishing a corresponding relationship of the identification information and the user of the apparatus 120; establishing a corresponding relationship of the user of the apparatus 120 and corresponding biological characteristic information; and establishing a corresponding relationship of the identification information and the biological characteristic information.

At block S303, displaying the identification information and the biological characteristic information in the webpage.

In at least one embodiment, the server 130 generates a bar code. One bar code is corresponding to one webpage. The webpage displays the identification information and the biological characteristic information.

At block S304, storing the identification information and the biological characteristic information.

In at least one embodiment, when the apparatus 120 determines that the identification information and the biological characteristic information are normal, the apparatus 120 transmits a determining instruction to the server 130. The server 130, in response to the determining instruction, stores the identification information and the biological characteristic information.

In another embodiment, when the apparatus 120 determines that the identification information and the biological characteristic information are abnormal, the apparatus 120 transmits a negative instruction to the server 130. The server 130, in response to the negative instruction, generates an abnormal alarm. The abnormal alarm includes displaying notification information in the webpage or outputting sound alarm by a speaker.

At block S305, refreshing the webpage to indicate whether the user of the apparatus 120 is allowed to enter the protection region.

In at least one embodiment, the server 130 refreshes the webpage including the notification information. The notification information includes the user of the apparatus 120 is allowed to enter the protection region or user of the apparatus 120 is not allowed to enter the protection region.

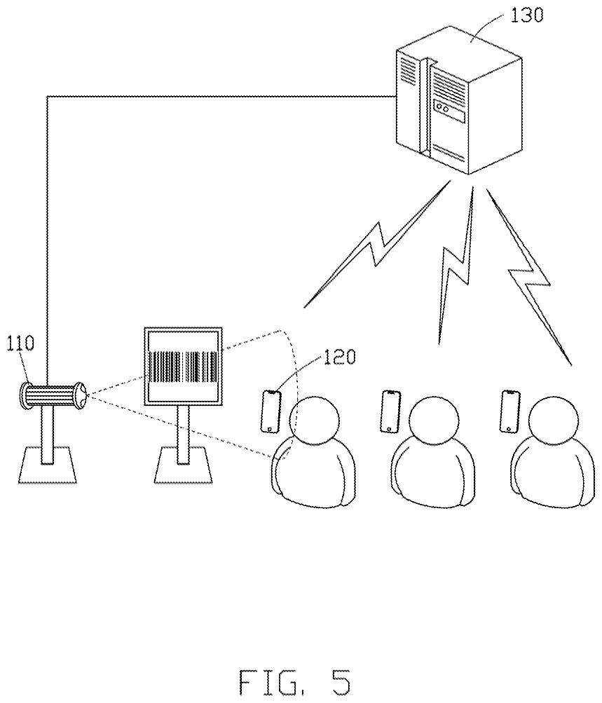

FIG. 5 is a schematic view of at least one embodiment of the system 100 for collecting information applied in a scenario.

Referring to FIG. 5, an entrance of the protection region includes a path. The people who wants to enter the protection region holds an apparatus 120 showing a bar code for being scanned, the apparatus 120 is linked to a webpage of the server 130. The server 130 acquires identification information from the apparatus 120 and biological characteristic information of the people from the collecting device 110. The server 130 matches the identification information and the biological characteristic information, and di splays the identification information and the biological characteristic information in the webpage. The apparatus 120 synchronously displays the content of the webpage, which includes the identification information, the biological characteristic information, the confirmation icon, and the negative icon. The people can browse the content of the webpage on the apparatus 120, if the identification information and the biological characteristic information are determined to be normal, touching or clicking the confirmation icon. If the identification information and the biological characteristic information are determined to be abnormal, touching or clicking the negative icon. If the identification information and the biological characteristic information are determined to be normal, the server 130 stores the identification information and the biological characteristic information, and refreshes the webpage to indicate the people is allowed to enter the protection region. If the identification information and the biological characteristic information are determined to be abnormal, the server 130 generates an abnormal alarm to indicate the people is not allowed to enter the protection region.

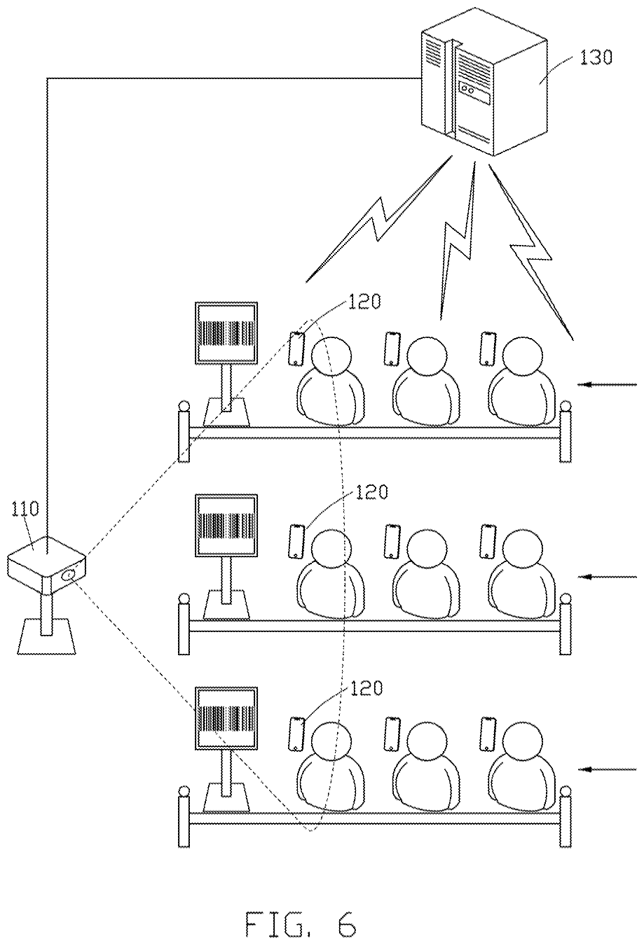

FIG. 6 is a schematic view of another embodiment of the system 100 for collecting information applied in a scenario.

Referring to FIG. 6, an entrance of the protection region includes a plurality of paths. Each path has a bar code. For instance, path k shows a kth bar code, the apparatus 120 can be linked to a kth webpage of the server 130 by scanning the kth bar code. The plurality of paths can synchronously collect information to improve an efficiency and decrease the workload of the entrance of the protection region. For each path, descriptions for the collecting device 110, the apparatus 120, and the server 130 can be the same with aforesaid embodiments, which is omitted here.

A non-transitory computer-readable storage medium including program instructions for causing the processor to perform the method for collecting information is also disclosed.

In at least one embodiment, the non-transitory computer-readable storage mediums can store local paths and computer instruction. The non-transitory computer-readable storage mediums can rapidly and automatically access instructions and data when the apparatus 120 and the server 130 is running. The non-transitory computer-readable storage mediums can be an internal storage system, such as a flash memory, a Random Access Memory (RAM) for the temporary storage of information, and/or a Read-Only Memory (ROM), a Programmable Read-Only Memory (PROM), Erasable Programmable Read-Only Memory (EPROM), a One-time Programmable Read-Only Memory (OTPROM), Electrically-Erasable Programmable Read-Only Memory (EEPROM), Compact Disc Read-Only Memory (CD-ROM) for permanent storage of information. The memory 31 can also be an external storage system, such as a hard disk, a storage card, or a data storage medium.

It is believed that the present embodiments and their advantages will be understood from the foregoing description, and it will be apparent that various changes may be made thereto without departing from the spirit and scope of the disclosure or sacrificing all of its material advantages, the examples hereinbefore described merely being embodiments of the present disclosure.

What is claimed is:

1. An apparatus for collecting information, the apparatus is communicated with a server and a collecting device, the apparatus comprises at least one processor and at least one memory, the at least one memory is coupled to the at least one processor and stores program instructions; the memory and the program instructions are configured to, with the at least one processor, cause the apparatus to perform:
scanning a bar code and transmit identification information of the apparatus to the server; wherein the bar code is configured to link to a webpage of the server;
acquiring biological characteristic information of a user of the apparatus collected by the collecting device;
acquiring the identification information and the biological characteristic information matched by the server; and
displaying the identification information and the biological characteristic information synced from the server.

2. The apparatus according to claim 1, wherein the memory and the program instructions configured to, with the at least one processor, cause the apparatus further to perform:
determining whether the identification information and the biological characteristic information are normal.

3. The apparatus according to claim 2, wherein the determining whether the identification information and the biological characteristic information are normal comprises:
displaying a confirmation icon;
detecting a confirmation operation on the confirmation icon from a user of the apparatus;
generating a determining instruction to determine whether the identification information and the biological characteristic information are normal; and
transmitting the determining instruction to the server.

4. The apparatus according to claim 3, wherein the determining whether the identification information and the biological characteristic information are normal comprises:
detecting no confirmation operation on the confirmation icon from the user; and
determining that the identification information and the biological characteristic information are abnormal.

5. A method for collecting information applied in an apparatus, the apparatus is communicated with a server and a collecting device, the method comprising:
scanning a bar code and transmit identification information of the apparatus to the server; wherein the bar code is configured to link to a webpage of the server;
acquiring biological characteristic information of a user of the apparatus collected by the collecting device;
acquiring the identification information and the biological characteristic information matched by the server; and
displaying the identification information and the biological characteristic information synced from the server.

6. The method according to claim 5, further comprising:
determining whether the identification information and the biological characteristic information are normal.

7. The method according to claim 6, wherein the determining whether the identification information and the biological characteristic information are normal comprises:
displaying a confirmation icon;
detecting a confirmation operation on the confirmation icon from a user of the apparatus;
generating a determining instruction to determine whether the identification information and the biological characteristic information are normal; and
transmitting the determining instruction to the server.

8. The method according to claim 7, wherein the determining whether the identification information and the biological characteristic information are normal comprises:
detecting no confirmation operation on the confirmation icon from the user; and
determining that the identification information and the biological characteristic information are abnormal.

9. A server for collecting information, the server is communicated with an apparatus and a collecting device, the server is configured to perform:
acquiring identification information of the apparatus from the apparatus and biological characteristic information of a user of the apparatus from the collecting device;
matching the identification information and biological characteristic information; and
displaying the identification information and biological characteristic information.

10. The server according to claim 9, wherein the matching the identification information and biological characteristic information comprises:
if determining the identification information and biological characteristic information being normal, storing the identification information and biological characteristic information; and
if determining the identification information and biological characteristic information being abnormal, generating an abnormal alarm.

11. The server according to claim 10, wherein the storing the identification information and biological characteristic information comprises:
refreshing the webpage to indicate whether a user of the apparatus being allowed to enter a protection region.

12. The server according to claim 9, wherein the matching the identification information and biological characteristic information comprises:
establishing a corresponding relationship of the identification information and the user of the apparatus;
establishing a corresponding relationship of the user of the apparatus and corresponding biological characteristic information; and
establishing a corresponding relationship of the identification information and the biological characteristic information.

13. The server according to claim 10, further comprising:
displaying a confirmation icon and a negative icon;
detecting a confirmation operation on the confirmation icon from a user and determining that the identification information and the biological characteristic information are normal; or
detects a negative operation on the negative icon from the user and determining that the identification information and the biological characteristic information are abnormal.

14. A method for collecting information applied in a server, the server is communicated with an apparatus and a collecting device, the method comprising:
acquiring identification information of the apparatus from the apparatus and biological characteristic information of a user of the apparatus from the collecting device;
matching the identification information and biological characteristic information; and
displaying the identification information and biological characteristic information.

15. The method according to claim 14, wherein the matching the identification information and biological characteristic information comprises:
if determining the identification information and biological characteristic information being normal, storing the identification information and biological characteristic information; and
if determining the identification information and biological characteristic information being abnormal, generating an abnormal alarm.

16. The method according to claim 15, wherein the storing the identification information and biological characteristic information comprises:
refreshing the webpage to indicate whether a user of the apparatus being allowed to enter a protection region.

17. The method according to claim 14, wherein the matching the identification information and biological characteristic information comprises:
establishing a corresponding relationship of the identification information and the user of the apparatus;
establishing a corresponding relationship of the user of the apparatus and corresponding biological characteristic information; and
establishing a corresponding relationship of the identification information and the biological characteristic information.

18. The method according to claim 15, further comprising:
displaying a confirmation icon and a negative icon;
detecting a confirmation operation on the confirmation icon from a user and determining that the identification information and the biological characteristic information are normal; or
detects a negative operation on the negative icon from the user and determining that the identification information and the biological characteristic information are abnormal.
